(19) United States
(12) Patent Application Publication
(10) Pub. No.: US 2010/0222875 A1
(43) Pub. Date: Sep. 2, 2010

(54) METHOD FOR FORMING A POROUS STENT COATING

(75) Inventor: Stephen Dirk Pacetti, San Jose, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 11/836,032

(22) Filed: Aug. 8, 2007

Related U.S. Application Data
(60) Provisional application No. 60/836,615, filed on Aug. 8, 2006.

(51) Int. Cl. A61F 2/06; A61F 13/00; A61K 31/195; A61K 31/445; A61K 31/56; A61K 38/00; A61P 43/00

(52) U.S. Cl. 623/1.49; 424/422; 427/2.3; 514/108; 514/178; 514/2; 514/326; 514/566

(57) ABSTRACT

Methods for forming porous stent coatings are disclosed.

### RELATED APPLICATION

This application claims benefit of and incorporates by reference U.S. Provisional Patent Application No. 60/836,615 which was filed on Aug. 8, 2006.

### FIELD OF THE INVENTION

The present invention is directed to a method for forming a porous stent coating.

### BACKGROUND OF THE INVENTION

The traditional method of administering therapeutic agents to treat diseases of the internal organs and vasculature has been by systemic delivery. Systemic delivery involves administering a therapeutic agent at a discrete location followed by the agent migrating throughout the patient's body including, of course, to the afflicted organ or area of the vasculature. But to achieve a therapeutic amount of the agent at the afflicted site, an initial dose substantially greater than the therapeutic amount must be administered to account for the dilution the agent undergoes as it travels through the body. Systemic delivery introduces the therapeutic agent in two ways: into the digestive tract (enteral administration) or into the vascular system (parenteral administration), either directly, such as injection into a vein or an artery, or indirectly, such as injection into a muscle or into the bone marrow. Absorption, distribution, metabolism, excretion and toxicology, the ADMET factors, strongly influence delivery by each of these routes. For enteric administration, factors such as a compound's solubility, its stability in the acidic environs of the stomach and its ability to permeate the intestinal wall all affect drug absorption and therefore its bioavailability. For parenteral delivery, factors such as enzymatic degradation, lipophilic/hydrophilic partitioning coefficient, lifetime in circulation, protein binding, etc. will affect the agent's bioavailability.

At the other end of the spectrum is local delivery, which comprises administering the therapeutic agent directly to the afflicted site. With localized delivery, the ADMET factors tend to be less important than with systemic administration because administration is essentially directly to the treatment site. Thus, the initial dose can be at or very close to the therapeutic amount. With time, some of the locally delivered therapeutic agent may diffuse over a wider region, but that is not the intent of localized delivery, and the diffused portion's concentration will ordinarily be sub-therapeutic, i.e., too low to have a therapeutic effect. Nevertheless, localized delivery of therapeutic agents is currently considered a state-of-the-art approach to the treatment of many diseases such as cancer and atherosclerosis.

Localized delivery of therapeutic agents may be accomplished using implantable medical devices, e.g., drug-eluting stents (DESs). In fact, DESs coated with antiproliferative and/or anti-inflammatory drugs are currently considered one of the most effective means of combating restenosis.

The efficacy of DESs is related to their ability to release drugs in a controlled manner. One way this is accomplished is by putting drugs in a drug reservoir layer that includes a polymeric matrix that mediates the release rate of the drug. Another way is to include on the DES a rate-controlling layer that is disposed over a drug reservoir layer and which comprises one or more polymers selected for their ability to mediate release of a particular drug or drugs from the underlying reservoir layer.

What is lacking in the art, however, are methods for producing DESs with reservoir layers and/or topcoat layers having pores, the interconnection of which is controllable and reproducible, to effectively allow drug release into the environment. The current invention provides such a means, devices incorporating those means and methods of using the devices for the treatment of a vascular disease.

### SUMMARY OF THE INVENTION

The present invention relates to a method for forming a porous coating on an implantable medical device. The method involves providing a device body, optionally disposing a primer layer over the device body, disposing a reservoir layer composition over the primer layer, if opted, or over the device body, where the reservoir layer composition includes one or more bioactive agents, disposing a topcoat layer composition over the reservoir layer, where the topcoat layer composition includes a first polymer, a first solvent miscible with the first polymer and second solvent immiscible with the first polymer, removing the first solvent from the topcoat layer composition and removing the second solvent from the topcoat layer composition, such that pores are formed in the resultant topcoat layer. The device body can be a stent.

In various aspects, the first polymer is selected from a group that includes poly(vinylidene fluoride), poly(vinylidene fluoride-co-hexafluoropropylene), poly(vinylidene fluoride-co-chlorotrifluoroethylene), poly(vinyl fluoride), poly(ethyl methacrylate), poly(propyl methacrylate), poly(butyl methacrylate), poly(methacrylates), poly(acrylates), poly(ethylene-co-vinyl acetate), poly(ethylene-co-vinyl alcohol), poly(vinyl acetate), poly(styrene-bl-isobutylene-bl-styrene), poly(styrene-bl-butylene-co-ethylene-bl-styrene), poly(L-lactide), poly(vinyl pyrrolidone), polyphosphorylcholinemethacrylate, PC1036, poly(D,L-lactide), poly(glycolide), poly(L-lactide-co-glycolide), poly(L-lactide-co-D,L-lactide), poly(D,L-lactide-co-glycolide), poly(caprolactone), poly(L-lactide-co-caprolactone), poly(D,L-lactide-co-caprolactone), poly(glycolide-co-caprolactone), poly(glycolide-bl-trimethylene carbonate-bl-glycolide), poly(esteramides), poly(anhydrides) and poly(tyrosine-carbonates).

In various aspects, the first solvent has a higher volatility than the second solvent.

In various aspects, the second solvent is selected from the group that includes water, propylene glycol, ethylene glycol, glycerol, benzyl alcohol, octanol, heptanol, hexanol, pentanol, butanol, propanol, ethanol, methanol, decalin, decane, nonane, octane, cyclooctane, heptane, cyclohexane, hexane, cyclopentane, pentane, carbon tetrachloride, freons, fluorinated solvents, perfluorinated solvents, tetrachloroethane and trichloroethane.

In various aspects, removing the first or second solvents comprises evaporation, lyophilization or supercritical extraction.

In various aspects, the volume fraction of the second solvent to the first polymer/second solvent blend is approximately 5% to 50%, approximately 15% to 50% or approximately 25% to 50%.

In various aspects, the resultant pores in the topcoat layer are discrete. In other aspects, the resultant pores in the topcoat layer are substantially interconnected.

In various aspects, the one or more bioactive agents are selected from the group that includes a corticosteroid, everolimus, an everolimus derivative, zotarolimus, a zotarolimus derivative, sirolimus, a sirolimus derivative, biolimus A9, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor, a tissue inhibitor of metalloproteinase, and combinations thereof.

In various aspects, the reservoir layer composition further includes a second polymer, a third solvent miscible with the second polymer and a fourth solvent immiscible with the second polymer.

In various aspects, the second polymer is independently selected from the same group of polymers as described above with respect to the first polymer.

In various aspects, the third solvent has a higher volatility than the fourth solvent.

In various aspects, the fourth solvent is independently selected from the same group of solvents as described above with respect to the second solvent.

In various aspects, the third solvent is removed from the reservoir layer composition by evaporation, lyophilization or supercritical extraction.

In various aspects, the fourth solvent is removed from the reservoir layer composition after the third solvent by evaporation, lyophilization or supercritical extraction, such that pores are formed in the resultant reservoir layer.

In various aspects, the volume fraction of the fourth solvent to the second polymer/fourth solvent blend is approximately 5% to 50%, is approximately 15% to 50% or is approximately 25% to 50%.

In various aspects, the resultant pores in the reservoir layer are discrete. In other aspects, the resultant pores in the reservoir layer are substantially interconnected.

Another aspect of the present invention relates to a method for forming a porous coating on an implantable medical device. The method involves providing a device body, optionally disposing a primer layer over the device body, disposing a reservoir layer composition over the primer layer, if opted, or over the device body, the reservoir layer composition including one or more bioactive agents, a polymer, a solvent miscible with the polymer and a solvent immiscible with the polymer, removing the miscible solvent from the reservoir layer composition and then removing the immiscible solvent from the reservoir layer composition, such that pores are formed in the resultant reservoir layer. The implantable medical device can be a stent.

In various aspects, the polymer is independently selected from the same group of polymers as described above with respect to the first and second polymers.

In various aspects, the miscible solvent has a higher volatility than the immiscible solvent.

In various aspects, the immiscible solvent is independently selected from the same group of solvents as described above with respect to the second and fourth solvents.

In various aspects, removing the miscible or immiscible solvents comprises evaporation, lyophilization or supercritical extraction.

In various aspects, the volume fraction of the immiscible solvent to the polymer/immiscible solvent blend is approximately 5% to 50%, approximately 15% to 50% or approximately 25% to 50%.

In various aspects, the resultant pores in the reservoir layer are discrete. In other aspects, the resultant pores in the reservoir layer are substantially interconnected.

In various aspects, the one or more bioactive agents are selected from the group of bioactive agents described above.

Another aspect of the present invention relates to an implantable medical device that includes a device body, an optional primer layer disposed over the device body, a reservoir layer disposed over the primer layer, wherein the reservoir layer is optionally porous and further includes one or more bioactive agents and an optional topcoat layer disposed over the reservoir layer, wherein the topcoat layer, if present, is optionally porous. In this aspect of the invention, at least one of the reservoir layer and topcoat layer must be porous. The implantable medical device can be a stent.

In various aspects, the primer layer includes poly(ester amide), poly(butyl methacrylate), poly(ethylene-co-vinyl alcohol), poly(vinyl alcohol) or poly(vinylidene fluoride-co-hexafluoropropylene).

In various aspects, the reservoir layer and the topcoat layer are independently selected from a group that includes poly(vinylidene fluoride), poly(vinylidene fluoride-co-hexafluoropropylene), poly(vinylidene fluoride-co-chlorotrifluoroethylene), poly(vinyl fluoride), poly(ethyl methacrylate), poly(propyl methacrylate), poly(butyl methacrylate), poly(methacrylates), poly(acrylates), poly(ethylene-co-vinyl acetate), poly(ethylene-co-vinyl alcohol), poly(vinyl acetate), poly(styrene-bl-isobutylene-bl-styrene), poly(styrene-bl-butylene-co-ethylene-bl-styrene), polyphosphorylcholinemethacrylate, PC1036, poly(vinyl pyrrolidone), poly(L-lactide), poly(D,L-lactide), poly(glycolide), poly(L-lactide-co-glycolide), poly(L-lactide-co-D,L-lactide), poly(D,L-lactide-co-glycolide), poly(caprolactone), poly(L-lactide-co-caprolactone), poly(D,L-lactide-co-caprolactone), poly(glycolide-co-caprolactone), poly(glycolide-bl-trimethylene carbonate-bl-glycolide), poly(esteramides), poly(anhydrides) and poly(tyrosine-carbonates).

In various aspects, the pores are discrete. In other aspects, the pores are substantially interconnected.

In various aspects, the one or more bioactive agents are selected from the group of bioactive agents described above.

Another aspect of the present invention relates to a method for treating or preventing a vascular disease involving implanting a medical device of the invention in a vessel of a patient in need thereof.

In various aspects, the vascular disease to be treated can be atherosclerosis, restenosis, vulnerable plaque or a peripheral arterial disease.

Another aspect of the present invention relates to an implantable medical device of the invention, where the primer layer includes poly(butyl methacrylate), the reservoir layer includes porous poly(butyl methacrylate) and everolimus and the topcoat layer includes poly(butyl methacrylate).

Another aspect of the present invention relates to an implantable medical device of the invention, where the primer layer includes poly(butyl methacrylate), the reservoir layer includes porous poly(butyl methacrylate) and everolimus and the topcoat layer includes porous poly(butyl methacrylate).

Another aspect of the present invention relates to an implantable medical device of the invention, where the primer layer includes poly(butyl methacrylate), the reservoir layer includes poly(butyl methacrylate) and everolimus and the topcoat layer includes porous poly(butyl methacrylate).

Another aspect of the present invention relates to an implantable medical device of the invention, where the primer layer includes poly(butyl methacrylate), the reservoir layer includes poly(butyl methacrylate) and everolimus and the topcoat layer includes porous poly(vinylidene fluoride-co-hexafluoropropylene).

Another aspect of the present invention relates to an implantable medical device of the invention, where the primer layer includes poly(butyl methacrylate), the reservoir layer includes poly(vinylidene fluoride-co-hexafluoropropylene) and everolimus and the topcoat layer includes porous poly(vinylidene fluoride-co-hexafluoropropylene).

Another aspect of the present invention relates to an implantable medical device of the invention, where the primer layer includes poly(butyl methacrylate) and the reservoir layer includes porous poly(vinylidene fluoride-co-hexafluoropropylene) and everolimus. In this aspect, there is no topcoat.

### DETAILED DESCRIPTION

Many strategies and coating configurations are used to control the drug dose and release rate of a DESs. The release rate is strongly affected by the nature of the drug and that of the polymer matrix. Only a relatively small number of polymers have proven to be suitable for drug eluting stent (DES) applications. Indeed, a particular polymer may be preferred specifically for its biological response and physical properties, since a desired drug, when combined with a preferred polymer, is often released too slowly from a DES. The release rate of a drug is also determined by the drug's diffusivity and solubility. Solubility is primarily a thermodynamic property, thus, modulating a drug's solubility requires changing the chemical properties of the drug or polymer. In contrast, diffusivity is a function of the drug's molecular size as well as the mesh size of the polymer.

Of import to the present invention is that a polymer's mesh size can be altered by the formation of pores within the polymer itself. One means of accomplishing this is to add such a large concentration of drug to the polymer that the drug phase separates out. Thus, during stent synthesis, drug is released, thereby forming pores within the polymer. However, a topcoat is often added to control drug release and prevent an initial bolus or burst release of drug. Unfortunately, sometimes even the thinnest topcoat that can be applied leads to a drug release that is too slow to be therapeutically effective. One example is everolimus in a PBMA drug coat with a PBMA topcoat. For example, it has been shown that a thin 40-μg PBMA topcoat on an 18-mm medium stent, i.e., a 0.3 micron coating thickness, with a drug:polymer ratio of 1 to 1.25, gave only 7.1% drug release over 24 hours in an in vitro experiment.

The present invention circumvents these issues by using pore-forming solvents in a coating composition. These pore-forming solvents are miscible with the coating composition for ease of processing but will be immiscible with the polymer. By judicious selection of solvent volatilities, the pore-forming solvent can be the slowest evaporating solvent in a polymer coating composition. This assures that at the end of the drying process, the pore-forming solvent is present and phase separates from the polymer matrix, thus forming pores.

The pore-forming solvent may be used in either the polymer/drug or topcoat layer coating compositions, and the volume fraction of the pore-forming solvent normalized to the polymer/pore-forming solvent blend should be in the range of 5-50% by volume. It is to be understood that once the lower volatility miscible solvent is removed, a two phase polymer/pore-forming solvent blend will remain. However, the presence of a pore-forming solvent does not necessarily mean that the coating surface will be porous. The surface porosity will be a function of the amount of pore-forming solvent and whether it migrates to the surface to minimize surface free energy during drying. At low pore forming solvent concentrations, for example, the voids formed will be discrete, while at high pore forming solvent concentrations the voids formed will interconnect.

The present invention relates to a method for forming a porous coating on an implantable medical device. The method involves providing a device body, optionally disposing a primer layer over the device body, disposing a reservoir layer composition over the primer layer, if opted, or over the device body, where the reservoir layer composition includes one or more bioactive agents, disposing a topcoat layer composition over the reservoir layer, where the topcoat layer composition includes a first polymer, a first solvent miscible with the first polymer and second solvent immiscible with the first polymer, removing the first solvent from the topcoat layer composition and removing the second solvent from the topcoat layer composition, such that pores are formed in the resultant topcoat layer.

As used herein, “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, PFO closure devices, arterial closure devices, artificial heart valves and cerebrospinal fluid shunts.

At present, preferred implantable medical devices for use with this invention are stents.

A stent refers generally to any device used to hold tissue in place in a patient's body. Particularly useful stents are those used for the maintenance of the patency of a blood vessel when the vessel is narrowed or closed due to diseases or disorders including, without limitation, tumors (in, for example, bile ducts, the esophagus or the trachea/bronchi), benign pancreatic disease, coronary artery disease, carotid artery disease, renal artery disease and peripheral arterial disease such as atherosclerosis, restenosis and vulnerable plaque. For example, a stent can be used to strengthen the wall of a vessel in the vicinity of a vulnerable plaque (VP). VP refers to a fatty build-up in an artery thought to be caused by inflammation. The VP is covered by a thin fibrous cap that can rupture leading to blood clot formation. Thus, a stent not only maintains vessel patency but can also act as a shield against such a rupture. A stent can be used in, without limitation, neuro, carotid, coronary, pulmonary, aortic, renal, biliary, iliac, femoral and popliteal as well as other peripheral vasculatures. A stent can be used in the treatment or prevention of disorders such as, without limitation, thrombosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. Indeed, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable. Due to the expansion of the stent, however, a stent coating must be flexible and capable of elongation.

Examples of stent materials include stainless steel, 316L, nitinol, tantalum, tantalum alloy, titanium, titanium alloy, cobalt chromium, L-605, Haynes 25, MP35N, nickel-titanium-platinum alloy, niobium, niobium alloy, zirconium and zirconium alloy.

As used herein, “device body” refers to a fully formed implantable medical device with an outer surface to which no coating or layer of material different from that of which the device itself is manufactured has been applied. “Outer surface” means any surface, however spatially oriented, that is in contact with bodily tissue or fluids. An example of a “device body” is a BMS, i.e., a bare metal stent, which is a fully-formed usable stent that has not been coated with a layer of any material different from the metal of which it is made. It is to be understood that device body refers not only to BMSs but also to any uncoated device regardless of what it is made.

As used herein, “optional” means that the element modified by the term may or may not be present. For example, without limitation, a device body (db) that has coated on it an “optional” primer layer (pl), a drug reservoir layer (dr) and a top-coat layer (tc) refers to either db+pl+dr+tc or db+dr+tc.

As used herein, “primer layer” refers to a coating consisting of a polymer or blend of polymers that exhibit good adhesion characteristics with regard to the material of which the device body is manufactured and good adhesion characteristics with regard to whatever material is to be coated on the device body. A primer layer is applied directly to a device body to serve as an intermediary layer between the device body and materials to be affixed to the device body. Examples, without limitation, of primers include silanes, titanates, zirconates, silicates, parylene, vinyl alcohol copolymers, acrylic acid copolymers, methacrylic acid copolymers, polyethyleneamine, polyallylamine, acrylate and methacrylate polymers with poly(n-butyl methacrylate).

As used herein, a material that is described as a layer “disposed over” an indicated substrate, e.g., a stent or another layer, refers to a relatively thin coating of the material applied directly to essentially the entire exposed surface of the indicated substrate. The term “disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “reservoir layer” refers to either a layer of one or more therapeutic agents applied to a medical device neat or alternatively to a layer of polymer or blend of polymers that has dispersed within its three-dimensional structure one or more therapeutic agents. A polymeric drug reservoir layer is designed such that, without limitation, by elution or as the result of biodegradation of the polymer, the therapeutic substance is released from the layer into the surrounding environment.

The reservoir layer generally comprises a biocompatible polymer that can be biostable or biodegradable and can be hydrophobic or hydrophilic. Suitable polymers are known to those skilled in the art.

As used herein, “biocompatible” refers to a polymer that both in its intact, as synthesized state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

In various aspects, the reservoir layer contains one or more bioactive agents which can be released from the medical device after implantation.

The bioactive agent, also referred to herein as a drug or a therapeutic agent, can be an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist or an antioxidant, or any combination thereof.

Examples of antiproliferative agents include, without limitation, actinomycins, taxol, docetaxel, paclitaxel, rapamycin, 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin, everolimus, biolimus, perfenidone and derivatives, analogs, prodrugs, co-drugs and combinations of any of the foregoing.

Examples of anti-inflammatory agents include both steroidal and non-steroidal (NSAID) anti-inflammatory agents such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecrolimus and derivatives, analogs, prodrugs, co-drugs and combinations of any of the foregoing.

Examples of antineoplastics and antimitotics include, without limitation, paclitaxel, docetaxel, methotrexate, azathioprine, vincristine, vinblastine, fluorouracil, doxorubicin hydrochloride, and mitomycin.

Examples of antiplatelet, anticoagulant, antifibrin, and antithrombin drugs include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax ä, calcium channel blockers such as nifedipine, colchicine, fish oil (omega 3-fatty acid), histamine antagonists, lovastatin, monoclonal antibodies such as those specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, nitric oxide or nitric oxide donors, super oxide dismutases, super oxide dismutase mimetic, 4-amino-2,2,6,6-tetramethylpiperidine-1-oxyl (4-amino-TEMPO) and derivatives, analogs, prodrugs, codrugs and combinations thereof.

Examples of cytostatic or antiproliferative agents include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Examples of antiallergic agents include, without limitation, permirolast potassium.

Other compounds that may be used as bioactive agents of this invention include, without limitation, alpha-interferon, genetically engineered epithelial cells, dexamethasone, antisense molecules which bind to complementary DNA to inhibit transcription, and ribozymes, antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy; antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and derivatives, analogs, prodrugs, codrugs and combinations of any of the foregoing.

Presently preferred bioactive agents include, but are not limited to, a corticosteroid, everolimus, zotarolimus, sirolimus, and derivatives and anologs thereof, biolimus A9, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase, and combinations thereof.

As used herein, “derivative” refers to a chemical compound having a modified structure of a base chemical compound.

As used herein, “analog” refers to a chemical compound that performs the same function as, but is a different chemical entity from, another chemical compound.

As used herein, “top-coat layer” refers to an outermost layer that is in contact with the external environment and that is disposed as the final layer of a series of layers.

A topcoat layer composition of the invention can include a first polymer, a first solvent miscible with the first polymer and a second solvent that is immiscible with the first polymer.

The first polymer can be selected from a group that includes, without limitation, poly(vinylidene fluoride), poly(vinylidene fluoride-co-hexafluoropropylene), poly(vinylidene fluoride-co-chlorotrifluoroethylene), poly(vinyl fluoride), poly(ethyl methacrylate), poly(propyl methacrylate), poly(butyl methacrylate), poly(methacrylates), poly(acrylates), poly(ethylene-co-vinyl acetate), poly(ethylene-co-vinyl alcohol), poly(vinyl acetate), poly(styrene-bl-isobutylene-bl-styrene), poly(vinyl pyrrolidone), poly(styrene-bl-butylene-co-ethylene-bl-styrene), polyphosphorylcholinemethacrylate, PC1036, poly(L-lactide), poly(D,L-lactide), poly(glycolide), poly(L-lactide-co-glycolide), poly(L-lactide-co-D,L-lactide), poly(D,L-lactide-co-glycolide), poly(caprolactone), poly(L-lactide-co-caprolactone), poly(D,L-lactide-co-caprolactone), poly(glycolide-co-caprolactone), poly(glycolide-bl-trimethylene carbonate-bl-glycolide), poly(esteramides), poly(anhydrides) and poly(tyrosine-carbonates). Based on the disclosure herein, one skilled in the art can ascertain first solvents with which the first polymer is miscible. The first solvent will be chosen so as to have a higher volatility than the second solvent.

The second solvent that is immiscible with the first polymer may be selected from a group that includes, without limitation, water, propylene glycol, ethylene glycol, glycerol, benzyl alcohol, octanol, heptanol, hexanol, pentanol, butanol, propanol, ethanol, methanol, decalin, decane, nonane, octane, cyclooctane, heptane, cyclohexane, hexane, cyclopentane, pentane, freons, fluorinated solvents, perfluorinated solvents, carbon tetrachloride, tetrachloroethane and trichloroethane.

In addition to being immiscible with a selected polymer, the second solvent will be chosen so as to be able to be loaded to an appropriate volume fraction in the polymer, thereby creating the potential to form voids, i.e., pores, in the polymer once the solvent is removed by a suitable process. Specifically, the ‘pore-forming’ solvent must be removable by a process that does not harm the drug or polymer. If it is simply a low-volatility solvent, then oven baking can effectuate removal. If not, processes such as lyophilization or supercritical extraction can remove the pore-forming solvent. Other suitable methods of removing the pore-forming solvent will be readily discernable to skilled practitioners in the art based on the disclosures herein.

As used herein, “volume fraction” refers to the volume of a referenced phase, i.e., immiscible solvent, with respect to the entire volume of material in a suspension of the invention, i.e., the immiscible solvent and polymer blend that remains after a miscible solvent is removed.

As used herein, “pore-forming solvent” refers to a solvent that has the ability to form pores in a polymer upon its removal from a solvent/polymer composition.

In a first aspect of the invention, a porous topcoat is formed over a bioactive agent reservoir layer on an implantable medical device. This occurs by disposing a topcoat layer composition over the reservoir layer, where the topcoat composition includes a first polymer, a first solvent miscible with the first polymer and a second solvent immiscible with the first polymer. It is to be understood that the composition will contain a sufficient amount of first solvent so that the first and second solvents will together be miscible with the first polymer when in the topcoat composition prior to the removal of the first solvent. Once the topcoat layer composition is disposed over the reservoir layer, the first solvent is removed by appropriate means as described above. Because the first solvent is chosen to have a higher volatility than the second solvent, it will be preferentially removed first. This will leave a first polymer and second solvent blend in which the second solvent is immiscible with the first polymer, thereby resulting in two phases which is necessary for the formation of pores upon removing the second solvent. Suitable pore-forming solvents are described above and will be chosen so as to have low volatility and poor solvency for the polymers of the present invention.

The second solvent is then removed by suitable methods, as described above, such that pores are formed in the resultant topcoat. The pores will be discrete or substantially interconnected depending on the volume fraction of the second solvent to the first polymer/second solvent blend.

As used herein, “discrete” refers to individual pores through which bioactive agent can diffuse.

As used herein, “substantially interconnected” refers to the situation in which at least 30% of the pores are connected to other pores thereby forming continuous channels through the polymer matrix through which bioactive agent can diffuse. It is well known at what volume fraction of immiscible solvent to a polymer/immiscible solvent blend pores begin to interconnect, thus the release rates of subsequently formed coatings will be controllable. In preferred embodiments, the volume fraction of the second solvent to the first polymer/second solvent blend will be approximately 5% to 50%, 15% to 50% or 25% to 50%.

In situations where the pores do not interconnect, the release rate will be affected to a smaller degree. However, if the pores formed do interconnect, the release rate can be dramatically increased. For example, if the goal is to have a high-molecular-weight drug, e.g., everolimus, diffuse through a fairly impermeable polymer, e.g., PBMA or poly-vinylidene fluoride (PVDF), then it may be necessary to have a high fraction of pores, or to have the pores interconnect. This is easily accomplished since it is well known at what solvent volume fraction pores begin to interconnect. Specifically, the transition from isolated pores to an interconnected pore network occurs over the range of 25-50 volume percent.

In addition to the presence of pores in the topcoat layer, the invention further provides for producing pores in the reservoir layer. In this aspect, the reservoir layer composition further includes a second polymer, a third solvent miscible with the second polymer and a fourth solvent immiscible with the second polymer. The second polymer can be the same as, or different from, the first polymer as listed above. In this aspect, the third solvent has a higher volatility than the fourth solvent which may be independently selected from the same group from which the second solvent is chosen.

Similar to the removal of the first solvent, as described above, the third solvent is removed from the reservoir layer composition by suitable means, as described above. Since the third solvent has a higher volatility than the fourth solvent, it will be preferentially removed first thereby leaving polymer and the immiscible fourth solvent. The fourth solvent will then be removed by suitable means as described above, such that pores are formed in the resultant reservoir layer.

The volume fraction of the fourth solvent to the second polymer/fourth solvent blend will be approximately 5% to 50%, 15% to 50% or 25% to 50% and the resultant pores in the reservoir layer will be discrete or substantially interconnected, as described above.

The present invention also provides a method for forming a porous reservoir layer on an implantable medical device absent a topcoat layer. In this aspect of the invention, the reservoir layer composition includes one or more bioactive agents, a polymer, a solvent miscible with the polymer and a solvent immiscible with the polymer. The polymer can be the same as the first and second polymers described above. In this aspect, the miscible solvent will have a higher volatility than the immiscible solvent, where the immiscible solvent is selected from the same group as the second and fourth solvents described above.

Similar to the removal of the first and third solvents, as described above, the miscible solvent is removed from the reservoir layer composition by evaporation, lyophilization or supercritical extraction. Since the miscible solvent has a higher volatility than the immiscible solvent, it will be preferentially removed first thereby leaving a polymer phase and an immiscible solvent phase. The immiscible solvent can then be removed by suitable means as described above, such that pores are formed in the resultant reservoir layer.

The volume fraction of the immiscible solvent to the polymer/immiscible solvent blend will be approximately 5% to 50%, 15% to 50% or 25% to 50% and the resultant pores in the reservoir layer will be discrete or substantially interconnected as described above.

The present invention also provides an implantable medical device that includes a device body, an optional primer layer disposed over the device body, a reservoir layer disposed over the primer layer, wherein the reservoir layer is optionally porous and further includes one or more bioactive agents, and an optional topcoat layer disposed over the reservoir layer, wherein the topcoat layer, if present, is optionally porous. In this aspect, the reservoir layer, the topcoat layer or both layers will be porous. In a preferred embodiment, the implantable medical device is a stent.

The primer layer can include, without limitation, poly(ester amide), poly(butyl methacrylate), poly(ethylene-co-vinyl alcohol), poly(vinyl alcohol) or poly-vinylidene fluoride-co-hexafluoropropylene. The reservoir layer and topcoat layers can include polymers independently selected from the group of polymers described above with respect to the first and second polymers.

In a preferred embodiment, an implantable medical device of the invention has a poly(butyl methacrylate) primer layer, a reservoir layer that includes porous poly(butyl methacrylate) and everolimus and a poly(butyl methacrylate) topcoat layer.

In another preferred embodiment, an implantable medical device of the invention has a poly(butyl methacrylate) primer layer, the reservoir layer includes porous poly(butyl methacrylate) and everolimus and the topcoat layer is porous poly(butyl methacrylate).

In another preferred embodiment, an implantable medical device of the invention has a poly(butyl methacrylate) primer layer, a reservoir layer that includes poly(butyl methacrylate) and everolimus and a porous poly(butyl methacrylate) topcoat layer.

In another preferred embodiment, an implantable medical device of the invention has a poly(butyl methacrylate) primer layer, a reservoir layer that includes poly(butyl methacrylate) and everolimus and a porous poly(vinylidene fluoride-co-hexafluoropropylene) topcoat layer.

In another preferred embodiment, an implantable medical device of the invention has poly(butyl methacrylate) primer layer, a reservoir layer that includes poly(vinylidene fluoride-co-hexafluoropropylene) and everolimus and a porous poly(vinylidene fluoride-co-hexafluoropropylene) topcoat layer.

In another preferred embodiment, an implantable medical device of the invention has a poly(butyl methacrylate) primer layer and a reservoir layer that includes porous poly(vinylidene fluoride-co-hexafluoropropylene) and everolimus but no topcoat layer.

The present invention also relates to a method of treating or preventing a vascular disease by implanting a medical device of the invention in a patient in need thereof. Methods of implanting medical devices are known to those skilled in the art.

### EXAMPLES

The following examples are provided to further teach the concepts and embodiments of the present invention. They are not intended nor are they to be construed in any manner to limit the scope of the present invention.

### Example 1

An 18-mm medium stent is coated with 50 μg of PBMA using a 2% solids solution in a 60/40 acetone/xylene mixture. After baking the stent at 80° C. for 0.5 hours, 675 μg of a 1:1.25 (w/w) everolimus/PBMA at a 2% solids solution, i.e., 300 μg drug, in the same solvent system is applied. After baking the stent at 80° C. for 0.5 hours, a pore-forming solvent containing topcoat composition consisting of 2% PBMA solids in a blend of 60/20/20 acetone/xylene/decane is applied. After applying 100 μg of the composition, the stent is baked at 80° C. for 1.0 hour. When immersed in blood, e.g., when placed in a blood vessel in vivo, the pores formed in the PBMA topcoat will allow everolimus to be released more rapidly than through a topcoat without any pores.

### Example 2

An 18-mm medium stent is coated with 50 μg of EVAL using a 2% solids solution in a 80/20 dimethyl acetamide (DMAC)/pentane mixture. After baking at 140° C. for 1.0 hour, 600 μg of 1:1.25 (w/w) clobetasol/EVAL at a 2% solids solution, in the same solvent system, is applied. After baking at 80° C. for 0.5 hours, a pore-forming solvent containing topcoat composition consisting of 2% PVDF-HFP solids in a blend of 40/30/30 cyclohexanone/acetone/decane is applied. After applying 150 μg of the composition, the stent is baked at 80° C. for one hour. When immersed in blood, e.g., when placed in a blood vessel in vivo, the pores formed in the PVDF-HFP topcoat allows clobetasol to be released more rapidly than through a topcoat without any pores.

### Example 3

An 18-mm medium stent is primed with 50 μg of EVAL from a 2% solids solution in a 80/20 blend of DMAC/pentane. After baking at 140° C. for 1.0 hour, a 750-μg drug coating of 1:1.5 paclitaxe/EVAL, i.e., 300 μg drug, at a 2% solids solution in DMAC/pentane/propylene glycol (70/10/20) (w/w) is sprayed onto the stent and baked at 80° C. for 2.0 hours. The propylene glycol forms pores in the EVAL, enabling paclitaxel to be released more rapidly than from a pure EVAL coating.

The present invention discloses a method for forming a porous coating on an implantable medical device using coating formulations that contain a solvent that forms pores in the polymer coating upon subsequent drying. The pore-forming solvents, i.e., the second, fourth and sixth solvents described above, are miscible with the coating formulation but immiscible with the polymer. Thus, by the judicious selection of solvent volatilities, the pore-forming solvent will be present at the end of the first drying stage, phase separate from the polymer matrix, and thus form pores upon drying. These pores will increase the diffusion rate of a drug through the polymer since drug diffusion rates are faster through a water-filled pore than through a polymer matrix. In certain situations, the pores will interconnect to form continuous channels through the polymer matrix, thereby greatly increasing the drug release rate. In various embodiments, the pores are formed in the topcoat layer, the drug reservoir layer or both layers, although the presence of pores in the topcoat layer is presently preferred. Suitable methods for forming these coatings are described below in the examples section.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention.

1. A method for forming a porous coating on an implantable medical device comprising:
providing a device body;
optionally disposing a primer layer over the device body;
disposing a reservoir layer composition over the primer layer, if opted, or over the device body to form a reservoir layer, the reservoir layer composition comprising one or more bioactive agents;
disposing a topcoat layer composition over the reservoir layer, the topcoat layer composition comprising a first polymer, a first solvent miscible with the first polymer and second solvent immiscible with the first polymer;
removing the first solvent from the topcoat layer composition;
removing the second solvent from the topcoat layer composition, such that pores are formed in the resultant topcoat layer;
wherein the first polymer is selected from the group consisting of poly(vinylidene fluoride-co-hexafluoropropylene), poly(vinylidene fluoride-co-chlorotrifluoroethylene), poly(ethyl methacrylate), poly(propyl methacrylate), poly(butyl methacrylate), poly(methacrylates), poly(acrylates), poly(ethylene-co-vinyl alcohol), poly(vinyl acetate), poly(styrene-bl-isobutylene-bl-styrene), poly(vinyl pyrrolidone), poly(styrene-bl-butylene-co-ethylene-bl-styrene), polyphosphorylcholinemethacrylate, PC1036, poly(L-lactide-co-D,L-lactide), poly(D,L-lactide-co-glycolide), poly(caprolactone), polv(L-lactide-co-caprolactone), poly(D,L-lactide-co-caprolactone), poly(glycolide-co-caprolactone), poly(glycolide-bl-trimethylene carbonate-bl-glycolide), poly(esteramides), and poly(tyrosine-carbonates).

2. (canceled)

3. The method according to claim 1, wherein the first solvent has a higher volatility than the second solvent.

4. The method according to claim 1, wherein the second solvent is selected from the group consisting of water, propylene glycol, ethylene glycol, glycerol, benzyl alcohol, octanol, heptanol, hexanol, pentanol, butanol, propanol, ethanol, methanol, decalin, decane, nonane, octane, cyclooctane, heptane, cyclohexane, hexane, cyclopentane, pentane, carbon tetrachloride, freons, fluorinated solvents, perfluorinated solvents, tetrachloroethane and trichloroethane.

5. The method according to claim 1, wherein removing the first or second solvent comprises evaporation, lyophilization or supercritical extraction.

6. The method according to claim 1, wherein the volume fraction of the second solvent to the first polymer/second solvent blend is approximately 5% to 50%.

7. The method according to claim 1, wherein the volume fraction of the second solvent to the first polymer/second solvent blend is approximately 15% to 50%.

8. The method according to claim 1, wherein the volume fraction of the second solvent to the first polymer/second solvent blend is 25% to 50%.

9. The method according to claim 1, wherein the resultant pores in the topcoat layer are discrete.

10. The method according to claim 1, wherein the resultant pores in the topcoat layer are substantially interconnected.

11. The method according to claim 1, wherein the one or more bioactive agents are selected from the group consisting of a corticosteroid, everolimus, an everolimus derivative, zotarolimus, a zotarolimus derivative, sirolimus, a sirolimus derivative, biolimus A9, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor, a tissue inhibitor of metalloproteinase, and combinations thereof.

12. The method according to claim 1, wherein the device body comprises a stent.

13. The method according to claim 1, wherein the reservoir layer composition further comprises a second polymer, a third solvent miscible with the second polymer and a fourth solvent immiscible with the second polymer wherein the reservoir layer composition comprises a sufficient amount of the third solvent so that the third and fourth solvents together are miscible with the second polymer.

14. The method according to claim 13, wherein the second polymer is selected from the group consisting of poly(vinylidene fluoride), poly(vinylidene fluoride-co-hexafluoropropylene), poly(vinylidene fluoride-co-chlorotrifluoroethylene), poly(vinyl fluoride), poly(ethyl methacrylate), poly(propyl methacrylate), poly(butyl methacrylate), poly(methacrylates), poly(acrylates), poly(ethylene-co-vinyl acetate), poly(ethylene-co-vinyl alcohol), poly(vinyl acetate), poly(styrene-bl-isobutylene-bl-styrene), poly(styrene-bl-butylene-co-ethylene-bl-styrene), polyphosphorylcholinemethacrylate, PC1036, poly(vinyl pyrrolidone), poly(L-lactide), poly(D,L-lactide), poly(glycolide), poly(L-lactide-co-glycolide), poly(L-lactide-co-D,L-lactide), poly(D,L-lactide-co-glycolide), poly(caprolactone), poly(L-lactide-co-caprolactone), poly(D,L-lactide-co-caprolactone), poly(glycolide-co-caprolactone), poly(glycolide-bl-trimethylene carbonate-bl-glycolide), poly(esteramides), poly(anhydrides) and poly(tyrosine-carbonates).

15. The method according to claim 13, wherein the third solvent has a higher volatility than the fourth solvent.

16. The method according to claim 13, wherein the fourth solvent is selected from the group consisting of water, propylene glycol, ethylene glycol, glycerol, benzyl alcohol, octanol, heptanol, hexanol, pentanol, butanol, propanol, ethanol, methanol, decalin, decane, nonane, octane, cyclooctane, heptane, cyclohexane, hexane, cyclopentane, pentane, freons, fluorinated solvents, perfluorinated solvents, carbon tetrachloride, tetrachloroethane and trichloroethane.

17. The method according to claim 13, wherein the third solvent is removed from the reservoir layer composition by evaporation, lyophilization or supercritical extraction.

18. The method according to claim 13, wherein the fourth solvent is removed from the reservoir layer composition after the third solvent by evaporation, lyophilization or supercritical extraction, such that pores are formed in the resultant reservoir layer.

19. The method according to claim 13, wherein the volume fraction of the fourth solvent to the second polymer/fourth solvent blend is approximately 5% to 50%.

20. The method according to claim 13, wherein the volume fraction of the fourth solvent to the second polymer/fourth solvent blend is approximately 15% to 50%.

21. The method according to claim 20, wherein the volume fraction of the fourth solvent to the second polymer/fourth solvent blend is approximately 25% to 50%.

22. The method according to claim 18, wherein the resultant pores in the reservoir layer are discrete.

23. The method according to claim 18, wherein the resultant pores in the reservoir layer are substantially interconnected.

24. A method for forming a porous coating on an implantable medical device comprising:
providing a device body;
optionally disposing a primer layer over the device body;
disposing a reservoir layer composition over the primer layer, if opted, or over the device body, the reservoir layer composition comprising one or more bioactive agents, a polymer, a solvent miscible with the polymer and a solvent immiscible with the polymer, wherein the reservoir layer composition comprises a sufficient amount of the miscible solvent so that the miscible and immiscible solvents together are miscible with the polymer;
removing the miscible solvent from the reservoir layer composition;
removing the immiscible solvent from the reservoir layer composition, such that pores are formed in the resultant reservoir layer.

25. The method according to claim 24, wherein the polymer is selected from the group consisting of poly(vinylidene fluoride), poly(vinylidene fluoride-co-hexafluoropropylene), poly(vinylidene fluoride-co-chlorotrifluoroethylene), poly(vinyl fluoride), poly(ethyl methacrylate), poly(propyl methacrylate), poly(butyl methacrylate), poly(methacrylates), poly(acrylates), poly(ethylene-co-vinyl acetate), poly(ethylene-co-vinyl alcohol), poly(vinyl acetate), poly(styrene-bl-isobutylene-bl-styrene), poly(styrene-bl-butylene-co-ethylene-bl-styrene), polyphosphorylcholinemethacrylate, PC1036, poly(vinyl pyrrolidone), poly(L-lactide), poly(D,L-lactide), poly(glycolide), poly(L-lactide-co-glycolide), poly(L-lactide-co-D,L-lactide), poly(D,L-lactide-co-glycolide), poly(caprolactone), poly(L-lactide-co-caprolactone), poly(D,L-lactide-co-caprolactone), poly(glycolide-co-caprolactone), poly(glycolide-bl-trimethylene carbonate-bl-glycolide), poly(esteramides), poly(anhydrides) and poly(tyrosine-carbonates).

26. The method according to claim 24, wherein the miscible solvent has a higher volatility than the immiscible solvent.

27. The method according to claim 24, wherein the immiscible solvent is selected from the group consisting of water, propylene glycol, ethylene glycol, glycerol, benzyl alcohol, octanol, heptanol, hexanol, pentanol, butanol, propanol, ethanol, methanol, decalin, decane, nonane, octane, cyclooctane, heptane, cyclohexane, hexane, cyclopentane, pentane, freons, fluorinated solvents, perfluorinated solvents, carbon tetrachloride, tetrachloroethane and trichloroethane.

28. The method according to claim 24, wherein removing the miscible or immiscible solvent comprises evaporation, lyophilization or supercritical extraction.

29. The method according to claim 24, wherein the volume fraction of the immiscible solvent to the polymer/immiscible solvent blend is approximately 5% to 50%.

30. The method according to claim 29, wherein the volume fraction of the immiscible solvent to the polymer/immiscible solvent blend is approximately 15% to 50%.

31. The method according to claim 30, wherein the volume fraction of the immiscible solvent to the polymer/immiscible solvent blend is approximately 25% to 50%.

32. The method according to claim 24, wherein the resultant pores in the reservoir layer are discrete.

33. The method according to claim 24, wherein the resultant pores in the reservoir layer are substantially interconnected.

34. The method according to claim 24, wherein the one or more bioactive agents are selected from the group consisting of a corticosteroid, everolimus, an everolimus derivative, zotarolimus, a zotarolimus derivative, sirolimus, a sirolimus derivative, biolimus A9, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor, a tissue inhibitor of metalloproteinase, and combinations thereof.

35. The method according to claim 24, wherein the device body comprises a stent.

36. An implantable medical device comprising:
a device body;
an optional primer layer disposed over the device body;
a reservoir layer disposed over the primer layer, wherein the reservoir layer is optionally porous and further comprises one or more bioactive agents; and
an optional topcoat layer disposed over the reservoir layer, wherein the topcoat layer is optionally porous, wherein at least one of the reservoir layer and topcoat layer must be porous.

37. The implantable medical device according to claim 36, wherein the device body comprises a stent.

38. The implantable medical device according claim 36, wherein the primer layer comprises poly(ester amide), poly(butyl methacrylate), poly(ethylene-co-vinyl alcohol), poly(vinyl alcohol) or poly(vinylidene fluoride-co-hexafluoropropylene).

39. The implantable medical device according to claim 36, wherein the reservoir layer and the topcoat layer are independently selected from the group that consists of poly(vinylidene fluoride), poly(vinylidene fluoride-co-hexafluoropropylene), poly(vinylidene fluoride-co-chlorotrifluoroethylene), poly(vinyl fluoride), poly(ethyl methacrylate), poly(propyl methacrylate), poly(butyl methacrylate), poly(methacrylates), poly(acrylates), poly(ethylene-co-vinyl acetate), poly(ethylene-co-vinyl alcohol), poly(vinyl acetate), poly(styrene-bl-isobutylene-bl-styrene), poly(styrene-bl-butylene-co-ethylene-bl-styrene), polyphosphorylcholinemethacrylate, PC1036, poly(vinyl pyrrolidone), poly(L-lactide), poly(D,L-lactide), poly(glycolide), poly(L-lactide-co-glycolide), poly(L-lactide-co-D,L-lactide), poly(D,L-lactide-co-glycolide), poly(caprolactone), poly(L-lactide-co-caprolactone), poly(D,L-lactide-co-caprolactone), poly(glycolide-co-caprolactone), poly(glycolide-bl-trimethylene carbonate-bl-glycolide), poly(esteramides), poly(anhydrides) and poly(tyrosine-carbonates).

40. The implantable medical device according to claim 36, wherein the pores are discrete.

41. The implantable medical device according to claim 36, wherein the pores are substantially interconnected.

42. The implantable medical device according to claim 36, wherein the one or more bioactive agents are selected from the group consisting of a corticosteroid, everolimus, an everolimus derivative, zotarolimus, a zotarolimus derivative, sirolimus, a sirolimus derivative, biolimus A9, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor, a tissue inhibitor of metalloproteinase, and combinations thereof.

43. A method for treating or preventing a vascular disease comprising implanting the medical device according to claim 36 in a vessel of a patient in need thereof.

44. The method according to claim 43, wherein the vascular disease is atherosclerosis, restenosis, vulnerable plaque or a peripheral arterial disease.

45. The implantable medical device according to claim 36, wherein
the primer layer comprises poly(butyl methacrylate);
the reservoir layer comprises porous poly(butyl methacrylate) and everolimus; and
the topcoat layer comprises poly(butyl methacrylate).

46. The implantable medical device according to claim 36, wherein
the primer layer comprises poly(butyl methacrylate);
the reservoir layer comprises porous poly(butyl methacrylate) and everolimus; and
the topcoat layer comprises porous poly(butyl methacrylate).

47. The implantable medical device according to claim 36, wherein
the primer layer comprises poly(butyl methacrylate);
the reservoir layer comprises poly(butyl methacrylate) and everolimus; and
the topcoat layer comprises porous poly(butyl methacrylate).

48. The implantable medical device according to claim 36, wherein
the primer layer comprises poly(butyl methacrylate);
the reservoir layer comprises poly(butyl methacrylate) and everolimus; and
the topcoat layer comprises porous poly(vinylidene fluoride-co-hexafluoropropylene).

49. The implantable medical device according to claim 36, wherein
the primer layer comprises poly(butyl methacrylate);
the reservoir layer comprises poly(vinylidene fluoride-co-hexafluoropropylene) and everolimus; and
the topcoat layer comprises porous poly(vinylidene fluoride-co-hexafluoropropylene).

50. The implantable medical device according to claim 36, wherein
the primer layer comprises poly(butyl methacrylate); and
the reservoir layer comprises porous poly(vinylidene fluoride-co-hexafluoropropylene) and everolimus, wherein there is no topcoat.
